(19) United States
(12) Patent Application Publication
(10) Pub. No.: US 2018/0177431 A1
(43) Pub. Date: Jun. 28, 2018

(54) IMPLANTABLE MONITORING DEVICE

(71) Applicant: HADASIT MEDICAL RESERACH SERVICES AND DEVELOPMENT LTD., Jerusalem (IL)

(72) Inventor: Yakir ROTTENBERG, Elazar (IL)

(21) Appl. No.: 15/580,568

(22) PCT Filed: Jun. 9, 2016
(86) PCT No.: PCT/IL2016/050601

§ 371 Date: Dec. 7, 2017

Related U.S. Application Data
(60) Provisional application No. 62/173,383, filed on Jun. 10, 2015.

(51) Int. Cl. A61B 5/053; A61B 5/00; A61B 10/02

(52) U.S. Cl. CPC A61B 5/0538; A61B 5/0531; A61B 5/0022; A61B 5/6848; A61B 5/4839; A61B 5/742; A61B 10/0233; A61B 5/4836

(57) ABSTRACT

There is provided herein an implantable device for monitoring a condition of a biological tissue, the device comprising: a sensor comprising a plurality of electrodes spaced apart from each other, an electric signal source configured to provide an electric signal to one or more pairs of neighboring or non-neighboring electrodes of said plurality of electrodes and an electric signal measurement unit configured to measure impedance values between each of said one or more pairs of electrodes wherein said signals produced by said electric signal measurement unit are indicative of a characteristic of a biological tissue adjacent the pair of electrodes.

### FIELD OF INVENTION

The present disclosure relates to a medical device for monitoring progression and augmentation of treatment of cancerous tissues. Some embodiments of the invention are directed to therapeutic treatment of solid tumors and causing necrosis of the cancer tissue.

### BACKGROUND

Cancer constitutes an enormous burden on society in more and less economically developed countries alike. The occurrence of cancer is increasing with the growth and aging of the population, as well as with the increasing prevalence of carcinogenic risk factors such as smoking, overweight, physical inactivity, and more. Based on GLOBOCAN estimates, about 14.1 million new cancer cases and 8.2 million deaths occurred in 2012 worldwide. Over the years, the burden has shifted to less developed countries, which currently account for about 57% of cancer cases and 65% of cancer deaths worldwide. (Torre et al. CA: A Cancer Journal for Clinicians, 2015, 65, 87-108). For example, breast cancer is the most common form of malignant disease among women in Western countries and, in the United States, is the most common cause of death among women between 40 and 55 years of age.

Monitoring cancer treatment and progression is challenging and typically requires expensive imaging techniques, such as PET and CT, which are typically employed at intervals of at least 2 months. Usually, these intervals are increased after a few cycles of treatment. There is thus a need for new techniques that would be sufficiently effective, on one hand, and not overly expensive, on the other hand, that would allow monitoring cancer treatment and/or progression.

Malignant neoplasms are abnormal tissues that exhibit different characteristics from those of normal tissues. For example, it is known that normal tissues have significantly higher electrical impedance than tumor tissues. Particularly, Morimoto, et al. (Eur. Surg. Res. 1990, 22, 86-92) reported measurable differences between impedance values of normal breast tissues, benign breast tumors and malignant breast tumors. Thus, electrical impedance measurements of body tissues can help distinguish cancerous from healthy tissues, as well as track cancer development or treatment.

For example, Eggers, in U.S. Pat. No. 5,630,426 discloses an apparatus for in situ diagnosis and treatment of tumor tissues, which enables differentiation among normal, malignant tumorous and nonmalignant tumorous biological tissues, by measuring the electrical impedance or the dielectric constant of the tissues. McRae, U.S. Pat. No. 5,069,223, discloses a method in which the electrical impedance of an identified tissue mass is used as a predictive assay of the progress of the hyperthermia treatment. In U.S. 2003/0009110 Tu describes a method of differentiating a tumorous tissue from a normal tissue by measuring the tissue's impedance values over a range of temperatures.

Surgical needles, such as trocar needles, are known and used during surgical procedures to access target body tissue or a target body cavity for observation, treatment, biopsy, and the like. The biopsy operation is a good opportunity to further examine internal physical characteristics of cancerous tissues. Stoianovici, in U.S. Pat. No. 6,337,994 provides a trocar needle, comprising an impedance probe that allows the surgeon to monitor the path of needle insertion, to confirm needle insertion into a desired anatomical target, and/or to identify the nature of cells surrounding the tip of the needle.

Despite the fact that previously described methods and apparatus allow detection and characterization of tissues within a specific time frame (e.g. during the course of a biopsy or another designated operation or imaging), a major deficiency still exists in the field of continuous monitoring of tissues over time.

### SUMMARY

The following embodiments and aspects thereof are described and illustrated in conjunction with systems, tools and methods which are meant to be exemplary and illustrative, not limiting in scope.

According to some embodiments, there is provided an implantable device (which may also be referred to as an implant) for monitoring a condition of a biological tissue, the device comprising a sensor comprising a plurality of electrodes spaced apart from each other, an electric signal source configured to provide an electric signal to one or more pairs of neighboring or non-neighboring electrodes of the plurality of electrodes and an electric signal measurement unit configured to measure impedance values between each of the one or more pairs of electrodes wherein the signals produced by the electric signal measurement unit are indicative of a characteristic of the biological tissue adjacent the pair of electrodes.

According to some embodiments, there is provided an implantable device for monitoring a condition of a biological tissue, the device includes a sensor including a plurality of electrodes spaced apart from each other; an electric signal source configured to provide an electric signal to one or more pairs of neighboring or non-neighboring electrodes of the plurality of electrodes; an electric signal measurement unit configured to measure impedance values between each of the one or more pairs of electrodes wherein the signals produced by the electric signal measurement unit are indicative of a characteristic of the biological tissue adjacent the pair of electrodes; and an anchoring element configured to anchor the implantable device to the biological tissue or to a neighboring tissue.

According to some embodiments, there is provided a system for monitoring a condition of a biological tissue. The system includes an implantable device for evaluation of a biological tissue and a processing circuitry unit. The device includes: a sensor comprising a plurality of electrodes spaced apart from each other; an electric signal source configured to provide an electric signal to one or more pairs of neighboring or non-neighboring electrodes of the plurality of electrodes; an electric signal measurement unit configured to measure impedance values between each of the one or more pairs of electrodes wherein the signals produced by the electric signal measurement unit are indicative of a characteristic of a biological tissue adjacent the pair of electrodes; and an anchoring element configured to anchor the implantable device to the biological tissue or to a neighboring tissue.

According to some embodiments, there is provided a kit for monitoring a condition of a biological tissue. The kit includes an implantable device for evaluation of a biological tissue and a biopsy needle. the device includes: a sensor comprising a plurality of electrodes spaced apart from each other; an electric signal source configured to provide an electric signal to one or more pairs of neighboring or non-neighboring electrodes of the plurality of electrodes; an electric signal measurement unit configured to measure impedance values between each of the one or more pairs of electrodes wherein the signals produced by the electric signal measurement unit are indicative of a characteristic of a biological tissue adjacent the pair of electrodes; and an anchoring element configured to anchor the implantable device to the biological tissue or to a neighboring tissue.

According to some embodiments, there is provided a method for monitoring a condition of a tumor, the method includes: a) using an impedance sensor implanted in the tumor of a subject, providing an electric signal to one or more pairs of neighboring or non-neighboring electrodes of a plurality of electrodes of the sensor; b) measuring, using an electric signal measurement unit, impedance values between each of the one or more pairs of electrodes wherein the signals produced by the electric signal measurement unit are indicative of a characteristic of a biological tissue within or in proximity to the tumor and adjacent the pair of electrodes; c) using a wireless transmitter, wirelessly transmitting the electric signals obtained from the electric signal measurement unit to processing circuitry outside the subject's body; and d) repeating steps a) and b) after a desired period of time, thereby monitoring the condition of the tumor.

According to some embodiments, the impedance may be electric impedance.

According to some embodiments, the electrical impedance of the biological tissue may be measured at one or more frequencies in the range of 20 kHz to 20 MHz.

According to some embodiments, the device may further be provided with a power source.

According to some embodiments, the device may further include a heating element configured to provide augmentation to a treatment provided to a subject having the tumor.

According to some embodiments, the device may further include a drug releasing component configured to release a drug to the tumor or the tumor's milieu.

According to some embodiments, the wireless transmitter may include a radio transmitter.

According to some embodiments, the wireless transmitter may include a Bluetooth transmitter.

According to some embodiments, the wireless transmitter may include a wireless passive indicator.

According to some embodiments, the wireless passive indicator may be configured to provide indication to a source outside the body of the subject with the tumor, corresponding to the signals from the electric signal measurement unit.

According to some embodiments, the wireless passive indicator may include electromechanical systems, such as Piezoelectric, RFID (radio frequency identification) or MEMS (micro-electromechanical systems).

According to some embodiments, the sensor (or additional sensor/s) may further be configured to measure pH, temperature, dielectric constant, capacitance, certain drug/s (for example, but not limited to, capecitabine) levels within the tumor microenvironment, or any combination thereof.

According to some embodiments, the sensor may further be configured to measure acoustic impedance, biochemical impedance, or both.

According to some embodiments, the biological tissue may include a tumor.

According to some embodiments, the tumor may be selected from a group consisting of: a solid tumor, a malignant tumor, an adenocarcinoma tumor, an adrenal gland tumor, an ameloblastoma tumor, an anaplastic tumor, an anaplastic carcinoma of the thyroid tumor, an angiofibroma tumor, an angioma tumor, an angiosarcoma tumor, an apudoma tumor, an argentaffmoma tumor, an arrhenoblastoma tumor, an astroblastoma tumor, an astrocytoma tumor, an ataxia-telangiectasia tumor, an atrial myxoma tumor, a basal cell carcinoma tumor, a benign tumor, a bone cancer tumor, a bone tumor, a brainstem glioma tumor, a brain tumor, a breast cancer tumor, a cancerous tumor, a carcinoid tumor, a carcinoma tumor, a cerebellar astrocytoma tumor, a cervical cancer tumor, a cherry angioma tumor, a cholangiocarcinoma tumor, a cholangioma tumor, a chondroblastoma tumor, a chondroma tumor, a chondrosarcoma tumor, a chorioblastoma tumor, a choriocarcinoma tumor, a colon cancer tumor, a craniopharyngioma tumor, a cystocarcinoma tumor, a cystofibroma tumor, a cystoma tumor, a cytoma tumor, a ductal carcinoma in situ tumor, a ductal papilloma tumor, a dysgerminoma tumor, an encephaloma tumor, an endometrial carcinoma tumor, an endothelioma tumor, an ependymoma tumor, an epithelioma tumor, an Ewing's sarcoma tumor, a feline sarcoma tumor, a fibro adenoma tumor, a fibro sarcoma tumor, a follicular cancer of the thyroid tumor, a ganglioglioma tumor, a gastrinoma tumor, an aglioblastoma multiform tumor, a glioma tumor, a gonadoblastoma tumor, an haemangioblastoma tumor, an haemangioendothelioblastoma tumor, an haemangioendothelioma tumor, an haemangiopericytoma tumor, an haematolymphangioma tumor, an haemocytoblastoma tumor, an haemocytoma tumor, a hamartoma tumor, an hepatocarcinoma tumor, an hepatocellular carcinoma tumor, an hepatoma tumor, an histoma tumor, a hypernephroma tumor, an infiltrating cancer tumor, an infiltrating ductal carcinoma tumor, an insulinoma tumor, a juvenile angioforoma tumor, a Kaposi sarcoma tumor, a kidney tumor, a lipoma tumor, a liver cancer tumor, a liver metastases tumor, a Lucke carcinoma tumor, a lung cancer tumor, a malignant mesothelioma tumor, a malignant teratoma tumor, a mastocytoma tumor, a medulloblastome tumor, a melanoma tumor, a meningioma tumor, a mesothelioma tumor, a metastatic tumor, a metastasis tumor, a metastatic spread tumor, a Morton's neuroma tumor, a myxoma tumor, a nasopharyngeal carcinoma tumor, a neoplastic tumor, a nephroblastoma tumor, a neuroblastoma tumor, a neurofibroma tumor, a neurofibromatosis tumor, a neuroglioma tumor, a neuroma tumor, an oligodendroglioma tumor, an optic glioma tumor, an osteochondroma tumor, an osteogenic sarcoma tumor, an osteosarcoma tumor, an ovarian cancer tumor, a Paget's disease of the nipple tumor, a pancoast tumor, a pancreatic cancer tumor, a phaeochromocytoma tumor, a pheoehromocytoma tumor, a primary brain tumor, a progonoma tumor, a prolactinoma tumor, a renal cell carcinoma tumor, a retinoblastoma tumor, a rhabdomyosarcoma tumor, a rhabdosarcoma tumor, a sarcoma tumor, a secondary tumor, a seminoma tumor, a skin cancer tumor, a small cell carcinoma tumor, a squamous cell carcinoma tumor, a strawberry haemangioma tumor, a teratoma tumor, a testicular cancer tumor, a thymoma tumor, a trophoblastic tumor, a tumorigenic tumor, a vestibular schwannoma tumor, and a Wilm's tumor. Each possibility represents a separate embodiment of the present disclosure.

According to some embodiments, the tumor may be selected from a group of: a solid tumor, a malignant tumor, a benign tumor, a brain tumor, a breast cancer tumor, a cancerous tumor, a carcinoid tumor, a carcinoma tumor, a colon cancer tumor, a cystoma tumor, a kidney tumor, a liver cancer tumor, a lung cancer tumor, a melanoma tumor, a metastatic tumor, a sarcoma tumor, a secondary tumor, a skin cancer tumor.

According to some embodiments, the sensor may be configured to be incorporated into a needle.

According to some embodiments, the needle may be selected from the group consisting of a biopsy needle and a trocar needle.

According to some embodiments, the processing circuitry unit may further include a user interface for providing an indication of at least one characteristic of the biological tissue.

According to some embodiments, the user interface may include a visual display monitor.

According to some embodiments, the visual display monitor may be adapted to displaying the at least one characteristic of the biological tissue in a manner adapted for evaluating the biological tissue by an operator.

According to some embodiments, the processing circuitry unit may further include an indication module configured to provide indication regarding an effectiveness of a treatment provided to the subject implanted with the implantable device.

According to some embodiments, the processing circuitry unit may further include a recommendation module configured to provide a recommendation regarding further treatment.

According to some embodiments, the method may include repeating steps a), b) and c) after a desired period of time.

According to some embodiments, the method may further include implanting the sensor essentially within the tumor.

According to some embodiments, the implantation may be performed during biopsy.

According to some embodiments, the method may further include providing treatment to the subject, wherein the treatment is intended to affect the tumor, and wherein steps a) and b) are performed at least once after the commencement of the treatment.

According to some embodiments, the desired period of time may be in a range of about 1 to 7 days.

According to some embodiments, the desired period of time may be in a range of about 7-90 days.

According to some embodiments, the method may further include augmenting treatment of the tumor using a heating element in or in conjugation with the sensor.

According to some embodiments, the heating may include local heating.

According to some embodiments the local heating may include elevating the temperature of regions of the biological tissue to a range of 40° C. to 90° C. (for example 60° C. to 80° C.).

According to some embodiments, the treatment augmentation may include causing necrosis of the tumor.

According to some embodiments, monitoring the condition of the tumor may include an on-line monitoring.

According to some embodiments, monitoring the condition of the tumor may include a continuous monitoring.

According to some embodiments, monitoring the condition of the tumor may include monitoring of the effectiveness of a treatment of the tumor.

According to some embodiments, the method may further include displaying the at least one characteristic of the tumor in a manner adapted for evaluating the tumor condition by an operator.

According to some embodiments, the method may include providing indication regarding an effectiveness of a treatment provided to the subject.

According to some embodiments, the method may further include providing a recommendation regarding further treatment.

According to some embodiments, the plurality of electrodes may include at least 2 electrodes.

According to some embodiments, the plurality of electrodes may include at least 3 electrodes.

According to some embodiments, the plurality of electrodes may include at least 5 electrodes.

According to some embodiments, the plurality of electrodes may include at least 10 electrodes.

According to some embodiments, the plurality of electrodes may include at least 20 electrodes.

According to some embodiments, the plurality of electrodes may include at least 50 electrodes.

According to some embodiments, the plurality of electrodes may include at least 100 electrodes.

According to some embodiments, the plurality of electrodes may include a two-dimensional, matrix-like array of electrodes.

According to some embodiments, the implantable device may further include an anchoring element, configured for fixing the device in or adjacent to the biological tissue, which is to be monitored during the monitoring period. The anchoring element is configured to prevent the dislocation of the implantable device during the monitoring period. According to some embodiments, the anchoring element may further be fastened and/or secured to the biological tissue during or after the implantation.

According to some embodiments, the anchoring element may include hooks and or spikes. According to some embodiments, the anchoring element may be deployed after the device was removed from the biopsy needle or from any other applicator.

According to some embodiments, the anchoring element may include an adhesive material. According to some embodiments, the fixing, fastening and/or securing may be performed by activating the adhesive material, for example by heat, radiation and/or by inducing any type of curing. According to some embodiments, the anchoring element may include a suture, a string, a wire and/or a thread, and the fixing, fastening and/or securing may be performed by suturing and/or tying the device to the region of interest, such as the tumor or a neighboring tissue. According to some embodiments, the anchoring element may include a clip, and the fixing, fastening and/or securing of the anchoring element may be performed by applying force and/or torsion, thus connecting the implant to the region of interest, such as the tumor or a neighboring tissue.

According to some embodiments, the anchoring element is configured to anchor the implantable device to a tissue, which is a neighboring tissue to the biological tissue to be monitored. According to some embodiments, the neighboring tissue is a tissue or an organ in proximity to the biological tissue to be monitored. According to some embodiments, the neighboring tissue may be a bone (for example, but not limited to, a skull bone, a rib, the sternum, a vertebra, or any other bone) or a connective tissue (for example, cartilage) in proximity to the biological tissue to be monitored.

According to some embodiments, the provided implantable device is configured to continuously monitor a condition of a biological tissue over time (for example, during hours, days, weeks or months), thus providing more meaningful data than a device which enables differentiation of normal tissues compared to cancerous tissues in a single point of time or during a certain procedure such as a biopsy.

According to some embodiments, the provided implantable device (or any processor related thereto) may be configured to provide integral analyses of a plurality of variables (for example, but not limited to, tumor type, specific drug, time from beginning of treatment and subject's age, gender and/or medical history) and the final result and/or output of the continuous monitoring may include a continuous variable of response (such as, but not limited to, ‘excellent’, ‘good’, ‘improved comparing to a previous time point’, etc.), rather than a dichotomic response (e.g. yes/no).

According to some embodiments, the implantable device may further provide information with a spatial resolution. In other words, the device may be configured to discriminate (differentiate) between a homogenous response to treatment and a heterogeneous response within the biological tissue. For example, the device may provide indication of necrosis at a specific region of the biological tissue (for example, at the center of a tumor), while indicating no or reduced effect on other regions of the biological tissue (for example, in peripheral areas of the tumor). According to some embodiments, such regional discrimination may also differentiate between sub-populations of cancer cells with distinctive features, such as, but not limited to, aggressiveness and/or responsiveness to certain treatment. In addition to the exemplary aspects and embodiments described above, further aspects and embodiments will become apparent by reference to the figures and by study of the following detailed description.

### BRIEF DESCRIPTION OF THE FIGURES

Exemplary embodiments are illustrated in referenced figures. Dimensions of components and features shown in the figures are generally chosen for convenience and clarity of presentation and are not necessarily shown to scale. It is intended that the embodiments and figures disclosed herein are to be considered illustrative rather than restrictive. The figures are listed below.

FIG. 1 schematically shows a biopsy assembly including a syringe and an implantable impedance sensing device inserted into the needle, according to some embodiments;

FIGS. 2a and 2b schematically show a subject having a breast tumor before implantation of an implantable impedance sensor (FIG. 2a) and after implantation of an implantable impedance sensor (FIG. 2b), according to some embodiments;

FIG. 3 schematically shows a block diagram of an implantable impedance sensing device, according to some embodiments;

FIG. 4 schematically shows a block diagram of an implantable impedance sensing device, according to some embodiments;

FIG. 5 schematically shows a block diagram of an impedance monitoring system, according to some embodiments;

FIG. 6 schematically shows a flow chart of a method for monitoring a condition of a tumor; and

FIGS. 7a -7g schematically show impedance values of tumors implanted with implantable impedance sensing devices vs. time, according to some embodiments.

### DETAILED DESCRIPTION

The following description relates to one or more non-limiting examples of embodiments of the invention. The invention is not limited by the described embodiments or drawings, and may be practiced in various manners or configurations or variations. The terminology used herein should not be understood as limiting unless otherwise specified.

The non-limiting section headings used herein are intended for convenience only and should not he construed as limiting the scope of the invention.

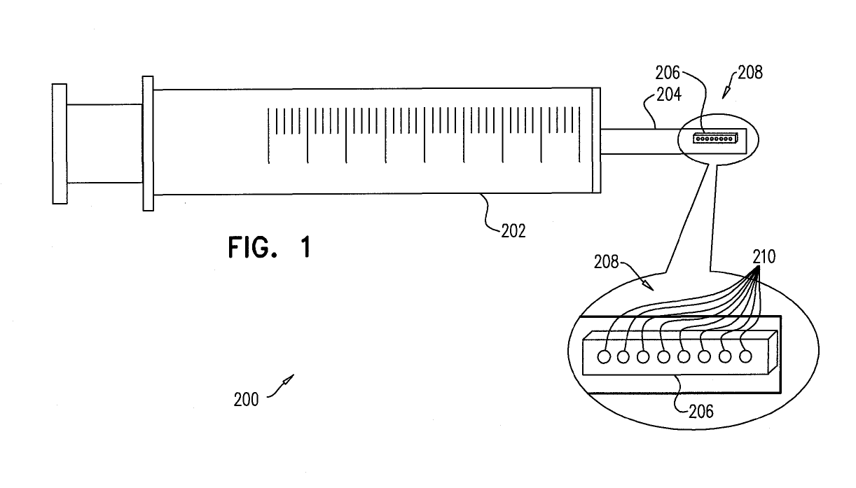

Reference is made to FIG. 1, which schematically shows a biopsy assembly 200 including a syringe 202 having a needle 204 and an implantable impedance sensing device 206 inserted into needle 204, according to some embodiments.

Implantable impedance sensing device 206 is configured to fit inside a lumen of biopsy needle 204. Implantable impedance sensor 206 is also shown, in needle 204, in an enlarged view 208. Implantable impedance sensing device 206 includes a plurality of isolated electrodes 210 and is configured to provide an electric signal corresponding to the electrical impedance between any pair of two electrodes of plurality of isolated electrodes 210.

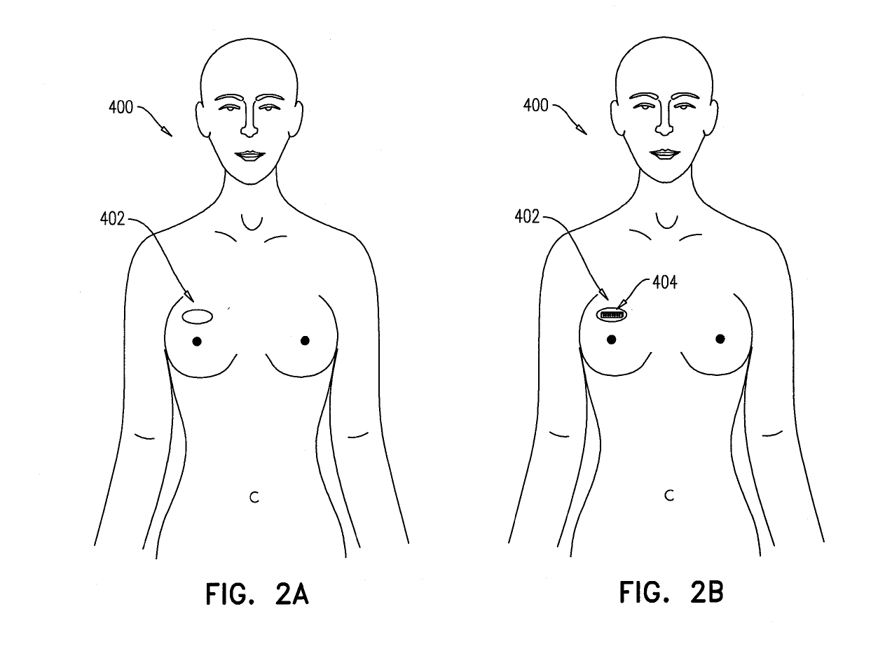

Reference is made to FIGS. 2a and 2b, which schematically show a subject having a breast tumor before implantation of an implantable impedance sensing device (FIG. 2a) and after implantation of an implantable impedance sensing device (FIG. 2b), according to some embodiments;

FIG. 2a represents a female subject 400 suffering from breast cancer, with a solid tumor 402 located inside her top right breast. FIG. 2b represents female subject 400, with solid tumor 402 located inside her top right breast after implantation of an implantable impedance sensing device 404. After implantation, implantable impedance sensing device 404 is located substantially in the center of solid tumor 402. According to some embodiments, implantable impedance sensing device 404 is configured to fit inside and be contained within solid tumor 402, as shown in FIG. 2b. It is noted that according to some embodiments, implantable impedance sensing device 404 may be only partially located within a tumor. As detailed herein, implantable impedance sensing device 404 is configured to provide signals corresponding to impedance, which provide indication relating to the tumor progression, remission and/or reaction to treatment.

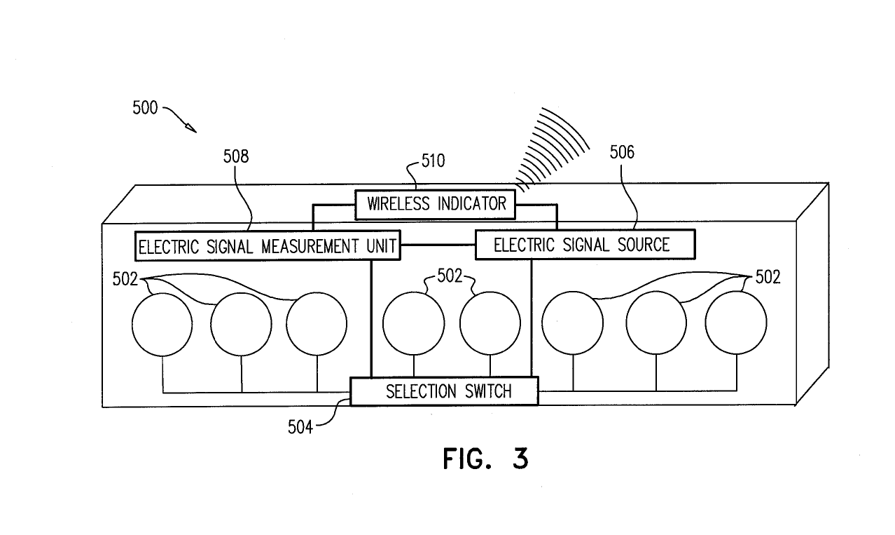

Reference is made to FIG. 3, which schematically shows a block diagram of an implantable impedance sensing device 500, according to some embodiments. According to some embodiments, implantable impedance sensing device 500 comprises a plurality of isolated electrodes 502 spaced apart from each other, which are logically connected to a single electric signal source 506 and to a single electric signal measurement unit 508 via a selection switch 504. It should be noted that the total number of electrodes may be any odd or even number higher than one (for example, 2-8, 3-10, 5-15, 10-100, 10-500 etc.), and not limited to a specific number as in the examples. It is also noted that plurality of isolated electrodes 502 may include a two-dimensional electrode, matrix-like array, in which each electrode is spaced apart from others.

Implantable impedance sensing device 500 is configured to provide an electric signal corresponding to the electrical impedance between any pair of two electrodes of plurality of isolated electrodes 502. According to some embodiments, to avoid stray capacitances, the electrodes may be connected via shielded wires to selection switch 504, which may select a specific pair of neighboring or non-neighboring electrodes, following a command from electric signal source 506. In order for implantable impedance sensing device 500 to provide an electric signal corresponding to the electrical impedance between a pair of two electrodes of plurality of isolated electrodes 502, electric signal measurement unit 508 is provided for measuring impedance values between a selected pair of electrodes of plurality of isolated electrodes 502. According to some embodiments, the signals are produced by electric signal measurement unit 508.

According to some embodiments, for the sake of allowing optimal monitoring of the tissue (such as tumor) condition, in which implantable impedance sensing device 500 is implanted, from outside subject body, a wireless indicator 510 is provided and configured to receive signal data from electric signal measurement unit 508 and from electric signal source 506. Wireless indicator 510 is further configured to provide wireless indication to a source outside the body, corresponding to the received signal data from electric signal measurement unit 508 and from electric signal source 506. According to some embodiments, wireless indicator 510 may be an active indicator, such as, but not limited to, a wireless transmitter configured to transmit signals in radio frequency as a wireless indication, for example, but not limited to, Bluetooth communication. According to some embodiments, wireless indicator 510 may be a passive indicator, which provides a passive physical indication, which can be detected from outside the body, by an appropriate apparatus, thus providing a wireless indication. Such passive indicators may include electromechanical systems, such as Piezoelectric, RFID (radio frequency identification) or MEMS (micro-electromechanical systems).

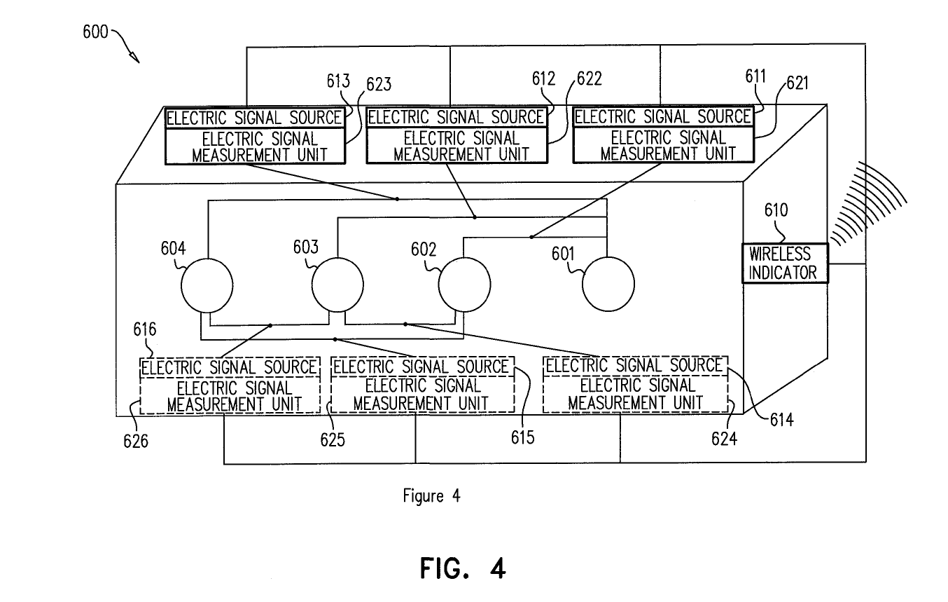

Reference is made to FIG. 4, which schematically shows a block diagram of an implantable impedance sensing device 600, according to some embodiments. According to some embodiments, implantable impedance sensing device 600 comprises a plurality of isolated electrodes 601-604 spaced apart from each other, and each pair of electrodes of plurality of isolated electrodes 601-604 is logically connected to a single electric signal source of electric signal sources 611-616 and to a single electric signal measurement unit of electric signal measurement units 621-626. It should be noted that the total number of electrodes may be any odd or even number higher than one (for example, 2-8, 3-10, 5-15, 10-100, 10-500 etc.), and not limited to a specific number as in the examples.

Implantable impedance sensing device 600 is configured to provide an electric signal corresponding to the electrical impedance between any pair of two electrodes of plurality of isolated electrodes 601-604. Electric signal sources 611-616 may supply electric current or voltage to the pair of electrodes of plurality of isolated electrodes 601-604, to which it is logically connected. In order for implantable impedance sensing device 600 to provide an electric signal corresponding to the electrical impedance between a pair of two electrodes of plurality of isolated electrodes 601-604, each one of electric signal measurement units 621-626 is configured to implantable impedance sensing device 600 for measuring impedance values between the pair of electrodes of plurality of isolated electrodes 601-604, to which it is logically connected. According to some embodiments, the signals are produced by electric signal measurement units 621-626.

According to some embodiments, for the sake of allowing optimal monitoring of the tissue (such as tumor) condition, in which implantable impedance sensing device 600 is implanted, from outside subject body a wireless indicator 610 is provided and configured to receive signal data from each one of electric signal measurement units 621-626 and from each one of electric signal sources 611-616. Wireless indicator 610 is further configured to provide wireless indication to a source outside the body, corresponding to the received signal data from each one of electric signal measurement units 621-626 and from each one of electric signal sources 611-616. According to some embodiments, wireless indicator 610 may be an active indicator, such as, but not limited to, a wireless transmitter configured to transmit signals in radio frequency as a wireless indication, for example, but not limited to, Bluetooth communication. According to some embodiments, wireless indicator 610 may be a passive indicator, which provides a passive physical indication, which can be detected from outside the body, by an appropriate apparatus, thus providing a wireless indication. Such passive indicators may include electromechanical systems, such as Piezoelectric, RFID (radio frequency identification) or MEMS (micro-electromechanical systems).

In an alternative configuration, also presented in FIG. 4, according to some embodiments, implantable impedance sensing device 600 includes a plurality of isolated electrodes 601-604 spaced apart from each other, and one electrode is selected as a reference electrode. It should be noted that the total number of electrodes may be any odd or even number higher than one (for example, 2-8, 3-10, 5-15, 10-100, 10-500 etc.), and not limited to a specific number as in the examples. For the sake of illustration in FIG. 4, isolated electrode 601 is selected as the reference electrode. Each isolated electrode, not selected as the reference electrode (isolated electrodes 602-604 in the example in FIG. 4) forms a primary pair together with the reference electrode (in the example in FIG. 4, the primary pair are formed from isolated electrodes; 601 and 602; 601 and 603; and 601 and 604). Electrode pairs which are not defined as primary pairs are defined as secondary pairs. For example, in the example of FIG. 4, secondary pairs are formed from isolated electrodes: 602 and 603; 602 and 604; and 603 and 604. Each primary pair is logically connected to a single electric signal source of electric signal sources 611-613 and to a single electric signal measurement unit of electric signal measurement units 621-623. In this alternative configuration, electric signal sources 614-616 and electric signal measurement units 624-626 in FIG. 4 may be absent. It should be noted that the total number of electric signal measurement units and electric signal sources may be any odd or even positive number (for example, 2-8, 3-10, 5-15, 10-100, 10-500 etc.), and not limited to a specific number as in the examples.

Also in the alternative configuration, implantable impedance sensing device 600 is configured to provide an electric signal corresponding to the electrical impedance between any pair of two electrodes of plurality of isolated electrodes 601-604, including both primary and secondary pairs. Electric signal sources 611-613 may supply electric current or voltage to the primary pairs of electrodes of plurality of isolated electrodes 601-604, to which it is logically connected. In order for implantable impedance sensing device 600 to provide an electric signal corresponding to the electrical impedance between a primary pair of two electrodes of plurality of isolated electrodes 601-604, each one of electric signal measurement units 621-623 is configured to implantable impedance sensing device 600 for measuring impedance values between the primary pair of electrodes of plurality of isolated electrodes 601-604, to which it is logically connected. According to some embodiments, the signals are produced by electric signal measurement units 621-623. Implantable impedance sensing device 600 may also provide an electric signal corresponding to the electrical impedance between a secondary pair of two electrodes of plurality of isolated electrodes 602-604. The impedance values between secondary pairs of electrodes of plurality of isolated electrodes 602-604 are calculated based on impedance values between the primary pair of electrodes of plurality of isolated electrodes 601-604. For example, in the alternative configuration of FIG. 4, the impedance values between isolated electrode 602 and isolated electrode 603 is calculated based on the impedance values between isolated electrode 601 and isolated electrode 602 and on the impedance values between isolated electrode 601 and isolated electrode 603.According to some embodiments of the alternative configuration, for the sake of allowing optimal monitoring of the tissue (such as tumor) condition, in which implantable impedance sensing device 600 is implanted, from outside subject body, a wireless indicator 610 is provided and configured to receive signal data from each one of electric signal measurement units 621-623 and from each one of electric signal sources 611-613. Wireless indicator 610 is further configured to provide wireless indication to a source outside the body, corresponding to the received signal data from each one of electric signal measurement units 621-623 and from each one of electric signal sources 611-613. According to some embodiments, wireless indicator 610 may be an active indicator, such as, but not limited to, a wireless transmitter configured to transmit signals in radio frequency as a wireless indication, for example, but not limited to, Bluetooth communication. According to some embodiments, wireless indicator 610 may be a passive indicator, which provides a passive physical indication, which can be detected from outside the body, by an appropriate apparatus, thus providing a wireless indication. Such passive indicators may include electromechanical systems, such as Piezoelectric, RFID (radio frequency identification) or MEMS (micro-electromechanical systems).

Implantable impedance sensing devices 500 (FIG. 3) or 600 (FIG. 4) may be used, for example, according to an impedance monitoring method, as part of an impedance monitoring system or an impedance monitoring kit, as described herein. Implantable impedance sensing devices 500/600 may be used in-vivo, for example, in conjunction with treating a patient; or may be used ex-vivo; or may be used externally to a human body, or without any relation to treating the human body. Optionally, the method, corresponding to implantable impedance sensing devices 500/600 may include calibrating implantable impedance sensing devices 500/600, or otherwise establishing baseline measurement value(s).

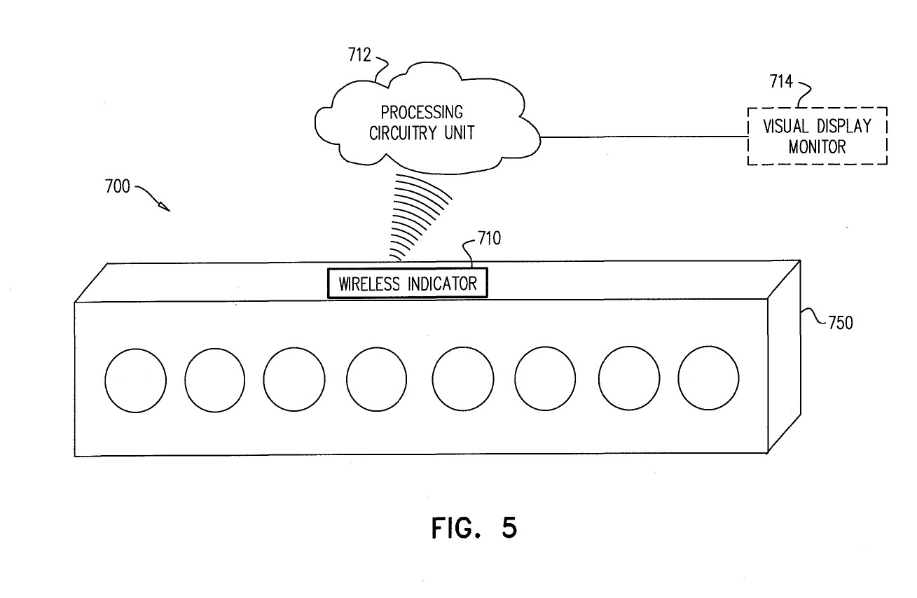

Reference is made to FIG. 5, which schematically shows a block diagram of an impedance monitoring system 700, according to some embodiments. According to some embodiments, impedance monitoring system 700 includes an implantable impedance sensing device 750, which includes a wireless indicator 710. According to some embodiments, wireless indicator 710 is configured to receive signal data indicative of a characteristic of a biological tissue (such as a tumor) into which implantable impedance sensing device 750 is implanted. According to some embodiments, wireless indicator 710 is further configured to provide wireless indication to a source outside the body, corresponding to the characteristic of a biological tissue into which implantable impedance sensing device 750 is implanted. According to some embodiments, wireless indicator 710 may be an active indicator, such as, but not limited to, a wireless transmitter configured to transmit signals in radio frequency as a wireless indication, for example, but not limited to, Bluetooth communication. According to some embodiments, wireless indicator 710 may be a passive indicator, which provides a passive physical indication, which can be detected from outside the body, by an appropriate apparatus, thus providing a wireless indication. Such passive indicators may include electromechanical systems, such as Piezoelectric, RFID (radio frequency identification) or MEMS (micro-electromechanical systems).

FIG. 5 depicts a case in which an active wireless transmitter is used as wireless indicator 710. It is configured to wirelessly transmit electric signals to a processing circuitry unit 712 corresponding to at least one characteristic of a biological tissue into which implantable impedance sensing device 750 is implanted. According to some embodiments, the characteristic is electrical impedance.

According to some embodiments, processing circuitry unit 712 may be configured to wirelessly receive and analyze signals obtained from wireless indicator 710. According to some embodiments, wireless indicator 710 is a wireless transmitter, and processing circuitry unit 712 is configured to periodically and regularly establish a wireless communication channel with wireless indicator 710 within an established communication channel. According to some embodiments, processing circuitry unit 712 is configured to periodically generate data values corresponding to the wireless signals received from wireless indicator 710 and indicative of at least one characteristic of a biological tissue into which implantable impedance sensing device 750 is implanted. Optionally, processing circuitry unit 712 operates in conjunction with a user interface, comprising a visual display monitor, 714, which is adapted to displaying impedance measurements of implantable impedance sensing device 750, in a manner adapted for evaluating the biological tissue by an operator.

According to some embodiments, a machine-learning algorithm is utilized for the monitoring of a condition of a tumor. According to some embodiments, the algorithm is configured to provide predictions for the diagnostic result in each case, the predictions having a probability of correctness factor. The prediction of the learning machine is then checked for correctness, and the algorithm is directed accordingly. In case the prediction turns out to be correct, the algorithm reinforces its calculation, thereby increasing the probability of the same prediction in similar future cases. In case the prediction turns out to be incorrect, the algorithm corrects its calculation, thereby decreasing the probability of the same prediction in similar future cases. The enforcement and correction mechanism described above may enable the algorithm to “learn” the behavior of cancerous tumors and provide a predicted diagnosis with high accuracy. According to some embodiments, the enforcement and correction mechanism is directed by a person (medical professional and/or computer professional). According to some embodiments, the enforcement and correction mechanism is directed by another algorithm, machine, computer, cloud, or the like, and/or any combination thereof. According to some embodiments, such an algorithm may include predetermined basic heuristics for detection of cancerous tumors and/or monitoring a condition of a tumor, for example, monitoring progression and/or augmentation of treatment of cancerous tissues. According to some embodiments, a pattern recognition algorithm is utilized. According to some embodiments, a computational learning algorithm is utilized. According to some embodiments, an artificial intelligence algorithm is utilized. According to some embodiments, one or more of the algorithms may be executed by the processing circuitry. According to some embodiments, one or more of the algorithms may be executed by an independent, remote and/or external processing circuitry, such as a remote server, a cloud server, a local computer and others.

Impedance monitoring system 700 may be used, for example, in conjunction with an impedance monitoring method or an impedance monitoring kit as described herein. The system may be used in-vivo, or in conjunction with treating a patient; or may be used ex-vivo; or may be used externally to a human body, or without any relation to treating the human body. Optionally, the method, corresponding to the impedance monitoring system, may include calibrating implantable impedance sensing device 750, or otherwise establishing baseline measurement value(s). The method may further include implanting implantable impedance sensing device 750 into a tissue in a human subject's body. According to some embodiments, the tissue is a tumor tissue, and according to some embodiments, the implantation is performed during biopsy.

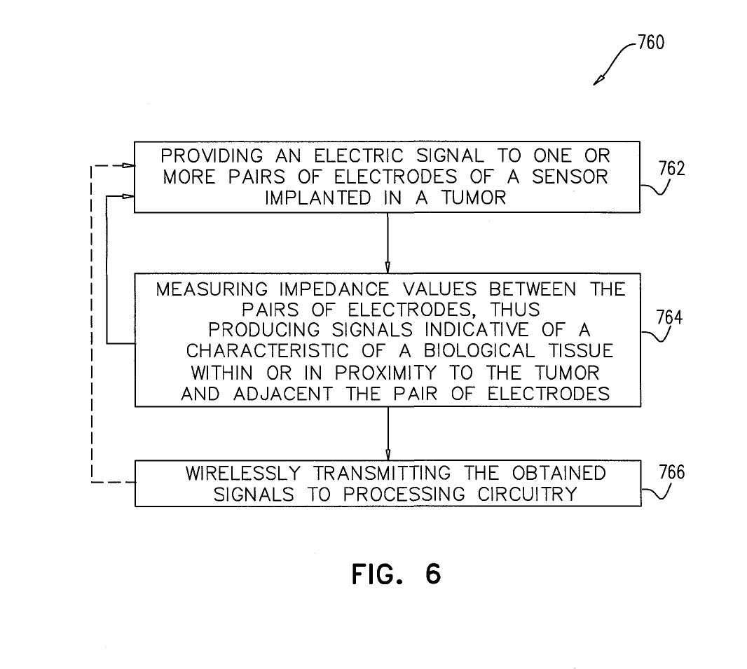

Reference is made to FIG. 6, which schematically shows a flow chart of a method for monitoring a condition of a tumor, 760, according to some embodiments. According to some embodiments, method 760 includes the following steps:

Step 762—providing an electric signal to one or more pairs of electrodes of a sensor implanted in a tumor. According to some embodiments, step 762 may be done using an impedance sensor. According to some embodiments, the one or more pairs of electrodes are one or more pairs of neighboring or non-neighboring electrodes of a plurality of electrodes of the sensor.

Step 764—measuring impedance values between the pairs of electrodes, thus producing signals indicative of a characteristic of a biological tissue within or in proximity to the tumor and adjacent the pair of electrodes. According to some embodiments, the measurements may be performed using an electric signal measurement unit. According to some embodiments, the electric signal measurement unit is producing the signals indicative of a characteristic of a biological tissue within or in proximity to the tumor and adjacent the pair of electrodes. According to some embodiments, the impedance values are measured between each of one or more pairs of electrodes of a plurality of electrodes.

Step 766—wirelessly transmitting the obtained signals to processing circuitry.

According to some embodiments, the transmitting is performed using a wireless transmitter. According to some embodiments, the obtained signals are electric signals obtained from the electric signal measurement unit. According to some embodiments, the processing circuitry is located outside the subject's body.

According to some embodiments, steps 762 and 764 are repeated after a desired period of time thereby monitoring the condition of the tumor. According to some embodiments, steps 762, 764 and 766 are repeated after a desired period of time, thereby monitoring the condition of the tumor.

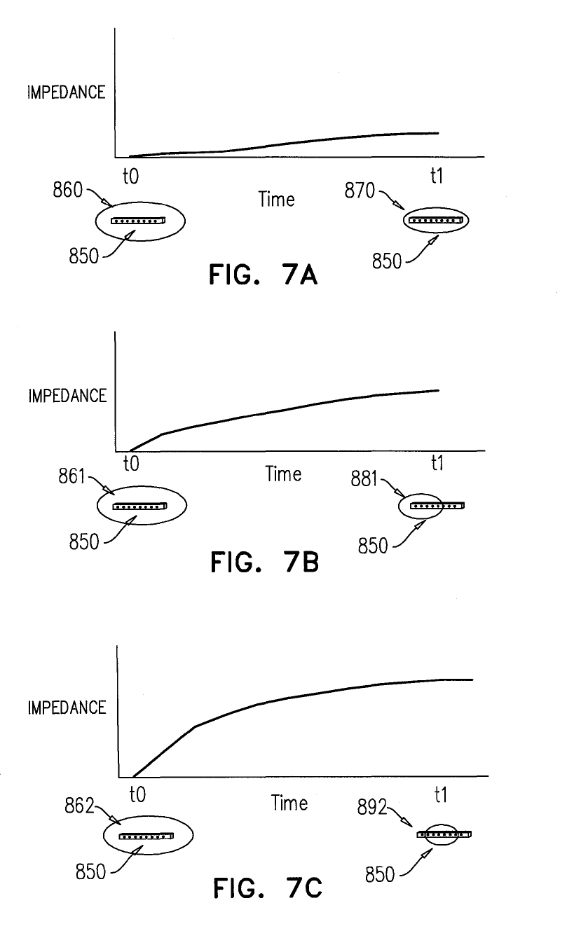

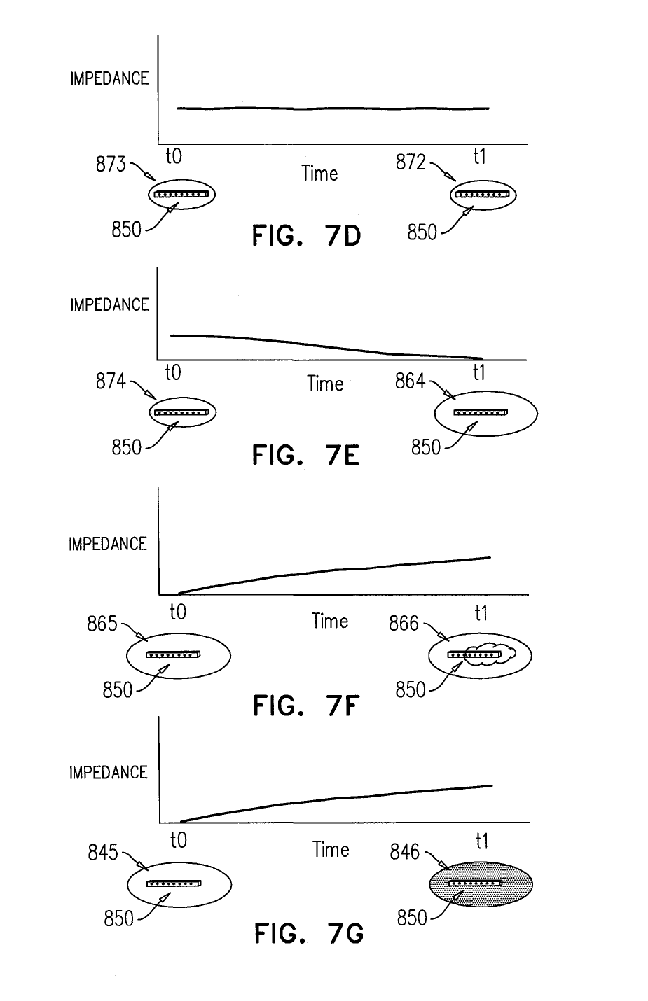

Reference is made to FIGS. 7a-7g, which schematically show impedance values around regions of tumors implanted with implantable impedance sensing devices 850 vs. time, according to some embodiments.

Without being bound by any theory or mechanism, normal tissues have higher (typically significantly higher) electrical impedance than tumor tissues, which can help monitoring cancer development or effects of treatment on the tumor. Consequently, a successful treatment of a tumor (such as malignant neoplasm), resulting in at least a partial necrosis or reduction in volume of the abnormal tissue, would also result in an increase of electrical impedance measured inside that tissue. Moreover, success of the treatment should be inversely proportional to the measured impedance.

For example, FIG. 7a shows an illustrative representation of measured tumor impedance vs. time, in a successful treatment, resulting in a moderate attenuation of the tumor size. At the beginning of the treatment, t=t0, the tumor is a large tumor, 860, depicted as a large ellipsoid, wholly containing an implantable impedance sensing device 850. Reference is made to time-point t=t1, which may represent any point of time in the duration or completion of the treatment, later than t=t0. At t=t1 the tumor, which represents the same tumor in t=t0, is now a medium tumor, 870 depicted as a medium ellipsoid, still wholly containing implantable impedance sensing device 850. According to the illustration depicted in FIG. 7a, the measured impedance is gradually increasing with time, and with tumor volume reduction due to the action of the successful treatment. Additionally, the moderate slope of the graph in FIG. 7a indicates a moderate decrease in tumor size between t=t0 and t=t1 corresponding to the moderate decrease in tumor size.

FIG. 7b is an illustrative representation of measured tumor impedance vs. time, in a successful treatment, resulting in a substantial attenuation of the tumor size. At the beginning of the treatment, t=t0, the tumor is a large tumor, 861, as in FIG. 7a, t=t0. Reference is made to time-point t=t1, which may represent any point of time in the duration or completion of the treatment, later than t=t0. At t=t1 the tumor, which represents the same tumor in t=t0, is now a small tumor, 881 depicted as a small ellipsoid, not large enough to contain implantable impedance sensing device 850. According to the illustration depicted in FIG. 7b, the measured impedance is gradually increasing with time, and with tumor volume reduction due to the action of the successful treatment. Additionally, the slope of the graph in FIG. 7b is steeper than the corresponding slope in FIG. 7a, indicating a more substantial decrease in tumor size between t=t0 and t=t1 in the case depicted in FIG. 7b than in the decrease in tumor size between t=t0 and t=t1 in the case depicted in FIG. 7a.

FIG. 7c is an illustrative representation of measured tumor impedance vs. time, in a successful treatment, resulting in a very significant reduction of the tumor size. At the beginning of the treatment, t=t0, the tumor is a large tumor, 862 as in FIGS. 7a and 7b (t=t0). Reference is made to time-point t=t1, which may represent any point of time in the duration or completion of the treatment, later than t=t0. At t=t1 the tumor, which represents the same tumor in t=t0, is now a very small tumor, 892 depicted as a very small ellipsoid, not large enough to contain implantable impedance sensing device 850. According to the illustration depicted in FIG. 7c, the measured impedance is gradually increasing with time, and with tumor volume reduction due to the action of the successful treatment. Additionally, the slope of the graph in FIG. 7c is steeper than the corresponding slopes in FIGS. 7a and 7b, indicating a more significant decrease in tumor size between t=t0 and t=t1 in the case depicted in FIG. 7c than in the decreases in tumor sizes between t=t0 and t=t1 in the cases depicted in FIGS. 7a and 7b.

Reference is made to FIG. 7d, which is an illustrative representation of measured tumor impedance vs. time, in a non-successful treatment, not resulting in visible change of tumor size. At the beginning of the treatment, t=t0, the tumor is a medium tumor, 873 as in FIG. 7a, t=t1. No visible change in tumor size is witnessed at time-point t=t1, which may represent any point of time in the duration or completion of the treatment, later than t=t0, and the tumor is still a medium tumor, 872. According to the illustration depicted in FIG. 7d, the measured impedance is practically constant with time, indicating an unchanged tumor volume between t=t0 and t=t1.

FIG. 7e is an illustrative representation of measured tumor impedance vs. time, in another unsuccessful treatment, resulting in an enlargement of the tumor size. At the beginning of the treatment, t=t0, the tumor is a medium tumor, 874 as in FIG. 7a, t =t1. Reference is made to time-point t=t1, which may represent any point of time in the duration or completion of the treatment, later than t=t0. At t=t1 the tumor, which represents the same tumor in t=t0, is now a large tumor, 864, as in FIG. 7a, t=t0.

According to the illustration depicted in FIG. 7e, the measured impedance is gradually decreasing with time, and with tumor enlargement. Additionally, the moderate slope of the graph in FIG. 7e indicative of a moderate increase in tumor size between t=t0 and t=t1.

Reference is made to FIGS. 7f and 7g, which are illustrative representations of measured tumor impedances vs. time, in successful treatments, resulting in necroses of the abnormal tissues. At the beginning of the treatments, t=t0, the tumors are non-necrotic large tumors, 865 (FIGS. 7f, t=t0) and 845 (FIG. 7g, t=t0). Reference is made to time-points t=t1 in FIGS. 7f and 7g, which may represent any points of time in the durations or completions of the treatments, later than t=t0 in FIGS. 7f and 7g. At t=t1 the tumors in FIGS. 7f and 7g, which represent the same tumors in t=t0 in FIGS. 7f and 7g, are now necrotic tumors, 866 (FIGS. 7f, t=t1) and 846 (FIG. 7g, t=t1), still wholly containing implantable impedance sensing devices 850. According to the illustration depicted in FIGS. 7f and 7g, the measured impedances are gradually increasing with time, and with the tumors' necroses due to the actions of the successful treatments.

While a number of exemplary aspects and embodiments have been discussed above, those of skill in the art will recognize certain modifications, permutations, additions and sub-combinations thereof. It is therefore intended that the following appended claims and claims hereafter introduced be interpreted to include all such modifications, permutations, additions and sub-combinations as are within their true spirit and scope.

Data may be analyzed by using a local or remote processing unit, processor, controller, Integrated Circuit (IC), system on a chip (SOC), workstation, portable electronic device, smartphone, tablet, laptop, general-purpose computing device, or other suitable device. Optionally, data processing may be performed live or in real-time by a server which may provide processing services to multiple or many units, based on a subscription fee, a pay-per-use fee, a pay-per-time-period subscription fee, or other suitable methods.

Some embodiments of the present disclosure may be implemented by utilizing any suitable combination of hardware components and/or software modules; as well as other suitable units or sub-units, processors, controllers, DSPs, CPUs, Integrated

Circuits, output units, input units, memory units, long-term or short-term storage units, buffers, power source(s), wired links, wireless communication links, transceivers, Operating System(s), software applications, drivers, or the like.

In the description and claims of the application, each of the words “comprise” “include” and “have”, and forms thereof, are not necessarily limited to members in a list with which the words may be associated.

1.-44. (canceled)

45. An implantable device for monitoring a condition of a biological tissue, the device comprising:
a sensor comprising a plurality of electrodes spaced apart from each other;
an electric signal source configured to provide an electric signal to one or more pairs of neighboring or non-neighboring electrodes of said plurality of electrodes;
an electric signal measurement unit configured to measure electrical impedance values between each of said one or more pairs of electrodes wherein said signals produced by said electric signal measurement unit are indicative of a characteristic of the biological tissue adjacent the pair of electrodes; and
an anchoring element configured to anchor the implantable device to the biological tissue or to a neighboring tissue.

46. The device according to claim 45, further comprising a wireless transmitter configured to wirelessly transmit said electric signals obtained from said electric signal measurement unit, wherein said wireless transmitter comprises a radio transmitter or a Bluetooth transmitter.

47. The device according to claim 45, further comprising a heating element configured to provide augmentation to a treatment provided to a subject having the tumor.

48. The device according to claim 45, further comprising a drug releasing component configured to release a drug to the tumor or the tumor's milieu.

49. The device according to claim 45, wherein said sensor is further configured to measure acoustic impedance, biochemical impedance, or both.

50. The device according to claim 45, wherein said biological tissue comprises a tumor.

51. The device according to claim 45, wherein said sensor is configured to be incorporated into a needle.

52. The device according to claim 51, wherein said needle is selected from the group consisting of a biopsy needle and a trocar needle.

53. A system for monitoring a condition of a biological tissue, the system comprising:
an implantable device for evaluation of a biological tissue, the device comprising:
a sensor comprising a plurality of electrodes spaced apart from each other;
an electric signal source configured to provide an electric signal to one or more pairs of neighboring or non-neighboring electrodes of said plurality of electrodes;
an electric signal measurement unit configured to measure impedance values between each of said one or more pairs of electrodes wherein said signals produced by said electric signal measurement unit are indicative of a characteristic of a biological tissue adjacent the pair of electrodes; and
an anchoring element configured to anchor the implantable device to the biological tissue or to a neighboring tissue; and
a processing circuitry unit configured to receive and analyze said electric signals obtained from said transmitter.

54. The system according to claim 53, wherein said processing circuitry unit further comprises a user interface for providing an indication of at least one characteristic of said biological tissue.

55. The system according to claim 53, wherein said processing circuitry unit further comprises a user interface for providing an indication of at least one characteristic of said biological tissue wherein said user interface comprises a visual display monitor, and wherein said visual display monitor is adapted to display said at least one characteristic of said biological tissue in a manner adapted for evaluating the biological tissue by an operator.

56. The system according to claim 53, wherein said processing circuitry unit further comprises an indication module configured to provide indication regarding an effectiveness of a treatment provided to the subject implanted with said implantable device.

57. A method for monitoring a condition of a tumor, the method comprising:
a) utilizing an impedance sensor implanted in the tumor of a subject, providing an electric signal to one or more pairs of neighboring or non-neighboring electrodes of a plurality of electrodes of the sensor;
b) measuring, utilizing an electric signal measurement unit, impedance values between each of said one or more pairs of electrodes wherein the signals produced by the electric signal measurement unit are indicative of a characteristic of a biological tissue within or in proximity to the tumor and adjacent the pair of electrodes; and
c) repeating steps a) and b) after a desired period of time thereby monitoring the condition of the tumor.

58. The method according to claim 57, further comprising implanting the sensor essentially within the tumor.

59. The method according to claim 57, further comprising providing treatment to the subject, wherein the treatment is intended to affect the tumor, and wherein steps a) and b) are performed at least once after the commencement of the treatment.

60. The method according to claim 57, wherein said tumor comprises a breast cancer tumor.

61. The method according to claim 57, wherein said tumor is selected from a group consisting of: a solid tumor, a malignant tumor, a benign tumor, a brain tumor, a breast cancer tumor, a cancerous tumor, a carcinoid tumor, a carcinoma tumor, a colon cancer tumor, a cystoma tumor, a kidney tumor, a leukemia tumor, a liver cancer tumor, a lung cancer tumor, a melanoma tumor, a metastatic tumor, sarcoma tumor, a secondary tumor, a skin cancer tumor.

62. The method according to claim 57, further comprising augmenting treatment of the tumor using a heating element in or in conjugation with the sensor, wherein heating comprises locally elevating the temperature of regions of said biological tissue to a range of 40° C. to 90° C.; and wherein the treatment augmentation comprises causing necrosis of said tumor.

63. The method according to claim 57, wherein monitoring the condition of the tumor comprises an on-line monitoring; and/or continuous monitoring.

64. The method according to claim 57, further comprising displaying the at least one characteristic of the tumor in a manner adapted for evaluating the tumor condition by an operator.
